(19) United States
(12) Patent Application Publication
(10) Pub. No.: US 2006/0122151 A1
(43) Pub. Date: Jun. 8, 2006

(54) Composition containing an association of aspirin and an anti-Xa oligosaccharide and use of an anti-Xa oligosaccharide optionally in combination with aspirin

(75) Inventor: Roger Guilcaume Cariou, Montpellier (FR)

Correspondence Address: JACOBSON HOLMAN PLLC, 400 SEVENTH STREET N.W., SUITE 600, WASHINGTON, DC, 20004, US

(73) Assignees: Sanofi-Synthelabo; Akzo Nobel NV.

(21) Appl. No.: 11/269,893

(22) Filed: Nov. 9, 2005

Related U.S. Application Data
(63) Continuation of application No. 10/155,221, filed on May 28, 2002.
Application No. 10/155,221 is a continuation of application No. 08/818,067, filed on Mar. 14, 1997, now abandoned.

(51) Int. Cl. A61K 31/737; A61K 31/60

(52) U.S. Cl. 514/54; 514/165; 514/61

(57) ABSTRACT

The invention relates to the use of a synthetic oligosaccharide which is a selective inhibitor of factor Xa acting via antithrombin III, alone or in association with aspirin, for the preparation of medicaments intended for preventing or treating thromboembolic diseases occurring in a mammal which has undergone a percutaneous transluminal angioplasty. The subject of the invention is moreover pharmaceutical compositions for the treatment or prophylaxy of thromboembolic diseases occurring in a mammal which has undergone a percutaneous transluminal angioplasty, comprising the association of an effective quantity of at least one synthetic oligosaccharide, which is a selective inhibitor of factor Xa acting via antithrombin III, and of an effective quantity of aspirin, optionally mixed with one or more pharmaceutically acceptable excipients.

The subject of the present invention is the use of synthetic oligosaccharides with factor Xa inhibiting activity which act via antithrombin III, alone or in combination with aspirin, in the treatment of thromboembolic diseases which occur during or after a percutaneous transluminal angioplasty (PTCA).

Pharmaceutical compositions containing the combination of the active ingredients oligosaccharides and aspirin are also part of the invention.

The active ingredients which constitute the combination are present in the free state or in the form of one of their pharmacologically acceptable salts.

During the last decade, a wide interest has been shown in the study of the role played by platelets in the development of the thromboembolic diseases associated with arteriosclerosis (myocardial infarction, angina, cerebral vascular accident, arterial diseases of the lower limbs and the like). Moreover, the well-established role of the blood coagulation process in arterial thrombosis has allowed the development of numerous medicaments which inhibit the various coagulation enzymes. The discovery of the essential role of thrombin and of factor Xa in the thrombotic process has led to the use of anticoagulants being proposed in the prevention and treatment of arterial thrombosis.

Among the available anticoagulants, heparin is the preferred medicament in the prevention and treatment of thromboembolic diseases.

Heparin catalyses, especially via antithrombin III (AT III), the inhibition of two enzymes which are involved in the blood coagulation cascade, namely factor Xa and factor IIa (or thrombin). The relative importance of these two activities in the overall activity of heparin remains unknown. Low molecular weight heparin (LMWH) preparations contain chains formed of 4 to 30 monosaccharides which act like heparin on factor Xa and on thrombin but which have the property of being more selective for factor Xa than thrombin. Despite this different biological activity profile, low molecular weight heparin has an antithrombotic effect as has been demonstrated in studies on animals and on patients suffering from thromboembolic diseases or exhibiting risks of formation of a thrombus (Hirsch J. et al, J. Thromb. Hemost., 1987, Leuven, Belgium Leuven University Press, 325-348).

Unlike heparin and the LMWHs, some synthetic oligosaccharides, especially those described in EP 84999, have the property of selectively inhibiting factor Xa via antithrombin III but do not possess any activity on thrombin.

These synthetic oligosaccharides corresponding to the antithrombin binding domain (ABD) of heparin are known and manifest an antithrombotic activity in venous thrombosis. These compounds are described in EP 529715 and EP 621282.

The efficacy of these oligosaccharides in the treatment of thromboembolic diseases occurring during or after percutaneous transluminal angioplasty was not very likely because of their incapacity to inhibit thrombin which is the mechanism involved in the thromboses resulting from a PTCA.

Indeed, it has long been known in the literature that thrombin plays a key role in arterial thrombosis and this is again confirmed by recent experiments (L. A. Harker, Blood, 1991, 77, 1006-1012). Thrombin inhibitors therefore constitute an effective means for preventing and combating this type of thrombosis after PTCA.

It has been observed, by comparing the efficacy of heparin with those of direct thrombin inhibitors (a direct inhibitor is an inhibitor which inhibits thrombin without requiring AT III as intermediate), that the latter are a lot more effective than heparin for preventing and treating arterial thrombosis (Arteriosclerosis and thrombosis, 1992, 12, 879-885. J. Am. Coll. Cardiol., 1994, 23, 993-1003). The probable reason for this lack of efficacy is that the heparin/AT III complex cannot, for reasons to do with stearic hindrance, inhibit thrombin in a platelet-rich thrombus as is a platelet thrombus.

The weak heparin activity, compared to the direct inhibitors, is therefore linked to its need to use AT III.

A compound which, on the one hand, acts itself via AT III as intermediate, and, on the other hand, does not inhibit thrombin would therefore be expected to be ineffective in the treatment of arterial thromboses after percutaneous transluminal angioplasty.

It has now been found, according to the present invention, quite surprisingly, that a synthetic oligosaccharide which is a selective inhibitor of factor Xa acting via antithrombin III, may be used alone or in combination with aspirin after PTCA in the treatment of thromboembolic diseases of arterial origin. Although it is now known that anti-factor Xa pentasaccharides and aspirin act via two different mechanisms of action, the combination or the association of these active ingredients for use as antithrombotics has never been studied.

Thus, according to one of its aspects, the subject of the present invention is the use of a synthetic oligosaccharide which is a selective inhibitor of factor Xa acting via AT III, alone or in combination with aspirin, for the preparation of medicaments intended for preventing or treating thromboembolic diseases occurring in a mammal which has undergone a percutaneous transluminal angioplasty.

According to the invention, selective inhibitor of factor Xa is understood to mean a compound capable of selectively inhibiting factor Xa via antithrombin III but not possessing a significant activity towards thrombin. Preferably the selective inhibitor of factor Xa has no activity towards thrombin.

Advantageously, the said synthetic oligosaccharides are pentasaccharides, such as those included in patents EP 84999 and U.S. Pat. No. 5,378,829.

Particularly advantageous pentasaccharides are especially:
methyl O-(2-deoxy-2-sulphoamino-6-O-sulpho-α-D-glucopyranosyl)-(1→4)-O-(β-D-glucopyranosyluronic acid)-(1→44)-O-(2-deoxy-2-sulphoamino-3,6-di-O-sulpho-α-D-glucopyranosyl)-(1→4)-O-(2-O-sulpho-α-L-idopyranosyluronic acid)-(1→4)-2-deoxy-2-sulphoamino-6-O-sulpho-α-D-glucopyranoside, in which the anion has the structure (A) 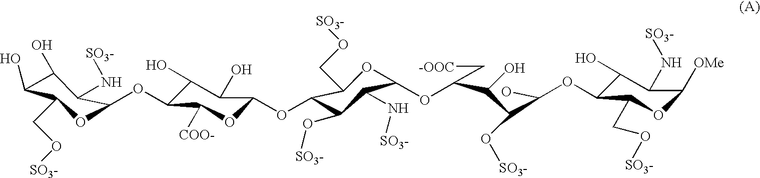
and its pharmaceutically acceptable salts, especially its decasodium salt, known by its code name SR 90107A or ORG 31540, described in Chemical Synthesis to Glycoaminoglycans, Supplement to Nature 1991, 350, 30-33, designated hereinafter “PS”.

Methyl O-(3,4-di-O-methyl-2,6-di-O-sulpho-α-D-glucopyranosyl)-(1→4)-O-(3-O-methyl-2-O-sulpho-β-D-glucopyranosyluronic acid)-(1→4)-O-(2,3,6-tri-O-sulpho-α-D-glucopyranosyl-(1→4)-O-(3-O-methyl-2-O-sulpho-α-L-idopyranosyluronic acid)-(1→4)-2,3,6-tri-O-sulpho-α-D-glucopyranoside, in which the anion has the structure (B) 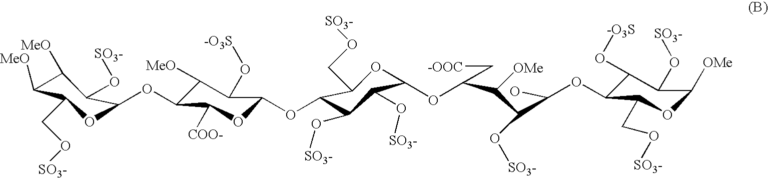
and its pharmaceutically acceptable salts, especially its dodecasodium salt described in U.S. Pat. No. 5,378,829;
Methyl O-(2,3,4-tri-O-methyl-6-O-sulpho-α-D-glucopyranosyl)-(1→4)-O-(2,3-di-O-methyl-β-D-glucopyranosyluronic acid)-(1→4)-O-(2,3,6-tri-O-sulpho-α-D-glucopyranosyl)-(1→4)-O-(2,3-di-O-methyl-α-L-idopyranosyluronic acid)-(1→4)-2,3,6-tri-O-sulpho-α-D-glucopyranoside, in which the anion has the structure (C) 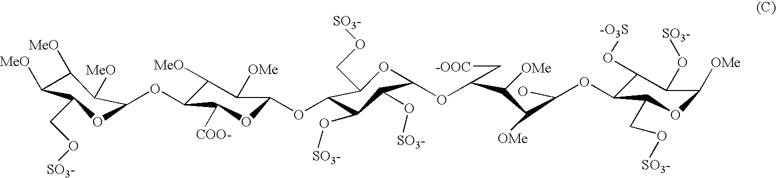
and its pharmaceutically acceptable salts, especially its nonasodium salt, also described in U.S. Pat. No. 5,378,829.

The use of the compound of formula (A), preferably in decasodium salt form (PS) corresponds to a preferred embodiment of the invention.

The decasodium salt of the compound of structure (A) (PS), as representative compound for use according to the present invention has been the subject of a pilot clinical study in patients undergoing a transluminal angioplasty of the coronaries. A single dose of 12 mg of PS via the intravenous route and 500 mg of aspirin, via the intravenous route, was administered to the patients. The results which were obtained show the therapeutic value of the PS/aspirin association in the prevention and treatment of acute thromboses following a percutaneous transluminal angioplasty.

To assess the antithrombotic activity 71 patients with stable angina (26), recent unstable angina (11) or recent myocardial infarction (34) with type A or B coronary lesions underwent PTCA with a single 12 mg PS i.v. bolus injection and 500 mg i.v. aspirin. Angiography was repeated 24 h after PTCA. The endpoints were thrombus formation at PTCA sites and the thrombolysis in myocardial infarction (TIMI) flow in target vessel. Heparin was not allowed before, during and within 24 h after PTCA. Acute thrombotic closure at dissected PTCA site occured in 1 patient and distal embolization of a thrombus containing plaque in 1 patient. Vessel patency was restored in both patients with intracoronary alteplase. Stents were required in 11 patients (for dissection in 9, suboptimal result in 2) who were given 250 mg ticlopidine at the time of implantation. Average minimal luminal diameter was 0.90±0.50 mm before and 2.65±0.40 mm after PTCA (reference diameter 2.95±0.60 mm). At 24 h TIMI 3 flow without thrombus at PTCA site was observed in all 71 patients. No major bleeding occurred. Anti-Xa activity peaked 10 min. after PS bolus (1.20±0.29 U anti-Xa/ml) and was maintained on average at 0.87±0.14 U anti-Xa/ml 2 hours after PS administration. The activated clothing time ACT remained unchanged. Thrombin-antithrombin complexes levels (TAT) fell from 21.9±18.7 to 4.8±3.8 μg/l and prothrombin fragment 1+2 from 2.08±1.04 to 1.54±82 ng/ml 2 hours after PS injection.

Thus the use according to the invention of an oligosaccharide, alone or in combination with aspirin is beneficial in relation to pathological states in patients having undergone a percutaneous transluminal angioplasty.

It will be noted that the use of the oligosaccharide, alone or in association with aspirin according to the invention, does not increase the haemorrhagic risk.

For the treatment of the abovementioned diseases, the oligosaccharide and the aspirin are administered to mammals, including man, at daily doses of the oligosaccharide or of the aspirin, respectively, of 0.1 to 100 mg per kilo of bodyweight of the mammal to be treated.

In a human being, the dose may vary for each of the components from 1 to 1000 mg per day, according to the age of the subject to be treated or the type of treatment: prophylactic or curative. Preferably, the pentasaccharide is administered at doses of between 0.30 mg and 30 mg per patient and per day.

The aspirin may be formulated in a pharmaceutical composition according to methods well known to persons skilled in the art. The same applies for the oligosaccharide.

The association of the oligosaccharide and the aspirin may be formulated in pharmaceutical compositions which may be used via the oral or parenteral route, especially via the subcutaneous or intravenous route, mixed with conventional pharmaceutical excipients.

These pharmaceutical compositions are preferably provided in the form of dosage units containing a predetermined quantity of active ingredients, such as for example from 0.1 to 50 mg of oligosaccharide or of aspirin, respectively, per dosage unit.

When at least two active ingredients are formulated in the same composition, it is necessary to ensure the compatibility of the different active substances. Thus the oligosaccharide is preferably used in the form of an addition salt, for example the sodium salt. Generally, indeed, the oligosaccharides in the form of their addition salts with pharmaceutically acceptable acids are not chemically incompatible with aspirin.

Thus, according to another of its aspects, the subject of the invention is a pharmaceutical composition for the treatment or prophylaxy of thromboembolic diseases in a mammal which has undergone a percutaneous transluminal angioplasty, comprising the association of an effective quantity of at least one synthetic oligosaccharide, which is a selective inhibitor of factor Xa acting via antithrombin III, and of an effective quantity of aspirin, optionally mixed with one or more pharmaceutically acceptable excipients.

These compositions are produced so as to be administrable via the digestive or parenteral route.

The pharmaceutical compositions of the invention are advantageously presented in various forms, such as for example injectable or oral solutions, sugar-coated tablets, plain tablets or gelatin capsules. The injectable solutions are the preferred pharmaceutical forms.

In the pharmaceutical compositions of the present invention for oral, sublingual, subcutaneous, intramuscular, intravenous, transdermal, transmucosal, local or rectal administration, the active ingredient may be administered in unit forms for administration, mixed with conventional pharmaceutical carriers, to animals and to human beings. The appropriate unit forms for administration comprise the oral forms such as tablets, gelatin capsules, powders, granules, microgranules and oral solutions or suspensions, the forms for sublingual and oral administration, the forms for subcutaneous, intramuscular, intravenous, intranasal or intraocular administration and the forms for rectal administration.

When a solid composition in tablet form is prepared, the principal active ingredient is mixed with a pharmaceutical vehicle such as gelatin, starch, lactose, magnesium stearate, talc, gum arabic and the like. The tablets may be coated with sucrose or other appropriate materials or they may be treated so that they have a prolonged or delayed activity and continuously release a predetermined quantity of active ingredient.

A preparation in gelatin capsules is obtained by mixing the active ingredient with a diluent and by pouring the mixture obtained into soft or hard gelatin capsules.

The water-dispersible powders or granules may contain the active ingredient mixed with dispersing agents or wetting agents, or suspending agents, such as polyvinylpyrrolidone, as well as with sweeteners or flavour correctors.

For a rectal administration, suppositories are used which are prepared with binders which melt at the rectal temperature, for example cocoa butter or polyethylene glycols.

For a parenteral, intranasal or intraocular administration aqueous suspensions, isotonic saline solutions and sterile and injectable solutions are used which contain dispersing agents and/or wetting agents which are pharmacologically acceptable, for example propylene glycol or butylene glycol.

For a transmucosal administration, the active ingredients may be formulated in the presence of a promoter such as a bile salt, a hydrophilic polymer such as for example hydropropyl cellulose, hydroxypropylmethyl cellulose, hydroxyethyl cellulose, ethyl cellulose, carboxymethyl cellulose, dextran, polyvinylpyrrolidone, pectins, starches, gelatin, casein, acrylic acids, acrylic esters and copolymers thereof, vinyl polymers or copolymers, vinyl alcohols, alkoxy polymers, polyethylene oxide polymers, polyethers or a mixture thereof.

The active ingredients may also be formulated in the form of microcapsules, optionally with one or more carriers or additives.

The active ingredients may also be provided in the form of a complex with a cyclodextrin, for example α- β- or γ-cyclodextrin, 2-hydroxypropyl-β-cyclodextrin, or methyl-β-cyclodextrin.

One of the active ingredients, for example the oligosaccharide, may also be released by a balloon containing it or by an endovascular stent introduced into the blood vessels. The pharmacological efficacy of the active ingredient is thus not affected.

According to a preferred embodiment of the invention, the pharmaceutical compositions comprise the association of aspirin and the compound of formula (A) preferably in the form of its decasodium salt.

Preferably still, the oligosaccharide is administered via the Intravenous or subcutaneous route.

The pharmaceutical compositions of the invention contain, preferably, from 5 to 30 mg of an oligosaccharide which is a selective inhibitor of factor Xa and 200 to 800 mg of aspirin, better still from 8 to 20 mg of the said oligosaccharide and from 400 to 600 mg of aspirin, for example 12 mg of the said oligosaccharide and 500 mg of aspirin.

1. Pharmaceutical composition for the treatment or prophylaxy of thromboembolic diseases in a mammal which has undergone a percutaneous transluminal angioplasty, comprising the association of an effective quantity of at least one synthetic oligosaccharide, which is a selective inhibitor of factor Xa acting via antithrombin III, and of an effective quantity of aspirin, optionally mixed with one or more pharmaceutically acceptable excipients.

2. Composition according to claim 1, comprising from 5 to 30 mg of the said oligosaccharide and from 200 to 800 mg of aspirin.

3. Composition according to claim 1, comprising from 8 to 20 mg of the said oligosaccharide and from 400 to 600 mg of aspirin.

4. Composition according to claim 1, wherein the oligosaccharide is
methyl O-(2-deoxy-2-sulphoamino-6-O-sulpho-α-D-glucopyranosyl)-(1→4)-O-(β-D-glucopyranosyluronic acid)-(1→4)-O-(2-deoxy-2-sulphoamino-3,6-di-O-sulpho-α-D-glucopyranosyl)-(1→4)-O-(2-O-sulpho-α-L-idopyranosyluronic acid)-(1→4)-2-deoxy-2-sulphoamino-6-O-sulpho-α-D-glucopyranoside, in which the anion has the structure (A) 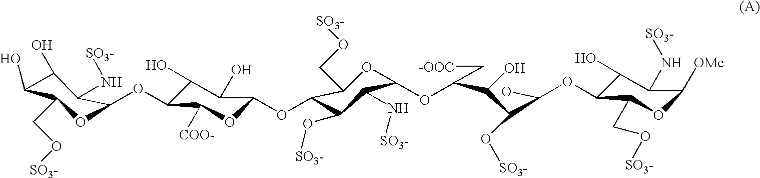
or one of its pharmaceutically acceptable salts.

5. Composition according to claim 4, wherein the oligosaccharide is the decasodium salt.

6. Process for the treatment or prophylaxy of thromboembolic diseases in a mammal having undergone a percutaneous transluminal angioplasty comprising the administration, to the said mammal, of an effective quantity of at least one synthetic oligosaccharide which is a selective inhibitor of factor Xa acting via antithrombin III, alone or in combination with aspirin.

7. Process according to claim 6, in which the oligosaccharide is methyl O-(2-deoxy-2-sulphoamino-6-O-sulpho-α-D-glucopyranosyl)-(1→4)-O-(β-D-glucopyranosyluronic acid)-(1→4)-O-(2-deoxy-2-sulphoamino-3,6-di-O-sulpho-α-D-glucopyranosyl)-(1→4)-O-(2-O-sulpho-α-L-idopyranosyluronic acid)-(1→4)-2-deoxy-2-sulphoamino-6-O-sulpho-α-D-glucopyranoside, in which the anion has the structure (A) 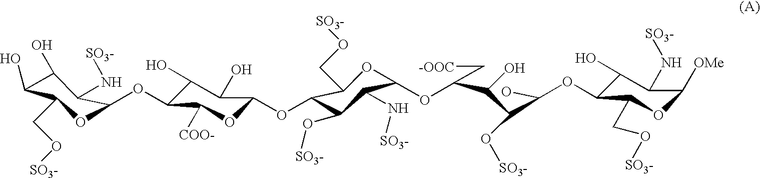
or one of its pharmaceutically acceptable salts.

8. Process according to claim 7, in which the oligosaccharide is the decasodium salt.

9. Process according to claim 6, comprising the administration of 0.1 to 100 mg of oligosaccharide per day and per kilo of bodyweight of the mammal to be treated.

10. Process according to claim 9, comprising, in addition, the administration of 0.1 to 100 mg of aspirin per day and per kilo of bodyweight of the mammal to be treated.

11. Process according to claim 6, characterized in that the oligosaccharide is administered via the intravenous or subcutaneous route.
